(19) United States
(12) Patent Application Publication
(10) Pub. No.: US 2015/0008336 A1
(43) Pub. Date: Jan. 8, 2015

(54) SHOE DEODARIZER AND SANITIZER

(71) Applicants: Yevgeny Rubinchikov, Redmond, WA (US); Serge Barlam, Redmond, WA (US)

(72) Inventors: Yevgeny Rubinchikov, Redmond, WA (US); Serge Barlam, Redmond, WA (US)

(21) Appl. No.: 13/936,070

(22) Filed: Jul. 5, 2013

(51) Int. Cl. A61L 2/10

(52) U.S. Cl. CPC A61L 2/10
USPC 250/455.11; 29/825

(57) ABSTRACT

The present invention is directed, in part, to an apparatus that effectively kills odor-causing bacteria within the interior of a shoe. The device doing so by employing use of a UV light source, housed within a rigid or semi-rigid framework, designed to fit snugly within the interior of a typical shoe. The framework of the device is designed to allow a maximum amount of UV light to illuminate and irradiate the interior of the shoe when the device is placed within the shoe. The device of the invention can be powered by means of an electrical adaptor, power supply or by battery power. In employing the device on a routine basis, a user can eliminate odor-causing bacteria and significantly increase the life of the shoes and effectively eliminate the embarrassment of odor from one's shoes.

### FIELD OF THE INVENTION

The present invention relates generally to an improved method and apparatus for sanitizing the interior of a shoe. More specifically, the invention relates to a lighting element and device that kills bacteria within the interior of a shoe, sanitizing the shoe and subsequently deodorizing the shoe, thus, providing added usefulness of the shoe, as well as a more pleasant experience, for the wearer.

### BACKGROUND OF THE INVENTION

Since the advent and use of shoes the issue of sweat and bacterial growth has been a recurring issue for humans. Foot odor due to the build up and growth of odor causing bacteria is often a constant source of embarrassment for shoe wearers. The cause of foot odor is well understood and stems from bacteria that grow on the bottom of feet. Foot dwelling bacteria consume molecules present in foot sweat and thereafter, as a by-product of consuming these molecules, the bacteria emit an unpleasant smelling gas that is detected by shoe wearers as foot odor.

Typically, the unpleasant smelling gas becomes a significant embarrassment to the shoe wearer leading to purchase of new shoes or for some remedy that will rid the shoes of the odor. Significantly, the business of shoe deodorizing is a billion dollar a year industry with consumers purchasing products ranging from Odor Eaters®, all manner of foot powders, shoe inserts and ultimately to complete replacement of the shoes. Similarly, the shoe industry has taken great pains to address the issue of foot odor and in killing the bacteria that lead to foot odor in the first place. The prior art is replete with all manner of remedies and devices that purportedly act to kill bacteria, viruses and other odor causing agents. For example, U.S. Pat. No. 8,186,004 discloses a UV source vacuum cleaner attachment for irradiation a cleaning surface in order to eradicate infesting agents such as molds, viruses, bacteria and dust mites. The device purportedly enhances penetration of UV light onto the desired surface by a variety of “mechanisms” employed with the device.

The prior art also provides numerous devices which purportedly use UV light as a mechanism for killing odor-causing bacteria in shoes. For example, United States Patent Application 2007/0164232 describes a device that uses ultraviolet C (UV-C) radiation and/or its by-product to sanitize and/or deodarize shoes. The device is held in place by a clip that attaches to the opening of the shoe and allows UV-C light to penetrate into the opening of the shoe. The light entering the shoe then purportedly santizes and deodarizes the interior of the shoe including the interior walls of the shoe.

Likewise, U.S. Patent Application 2002/0083535 discloses a device for deodarizing and disinfecting a shoe by means of irradiation of biocidal ultraviolet light (UV). The light includes a tubular light pipe that at one end incorporates a UV lamp connected to a power source, while the opposite end is inserted into the shoe. The inserted end may be covered by a protective cover made of thin polymer film that is essentially transparent in the UV spectral range. As light permeates the interior of the shoe, the device then purportedly kills odor-causing bacteria.

U.S. Pat. No. 8,226,887 discloses methods, devices and materials for a mobile device that provides ultraviolet light in order to santize or sterilize the surface of an object. The patent further discloses the device wherein a user moves the light source across the surface of an object in order to sanitize the object. The device is further described as being operatively coupled to a microprocessor that receives the movement data so that the amount of irradiation can be measured and a predetermined dosage of UV light sent onto the object.

U.S. Pat. No. 7,341,695 discloses an “anti-fouling apparatus” and methods that includes a UV light that is connected to an object or surface, the light providing a UV source, to prevent the growth of “foulants” on the object or surface. Also included with the device is a camera that is able to view the object as the cleaner removes the foulants from the surface or object. Such devices, although useful in some large-scale applications, do not address the shortcomings in the art related to eradication of foot odor from shoes.

U.S. Pat. No. 4,896,042 discloses a device with a combination of a vacuum cleaner and germicidal light source for killing bacteria that is moved by hand over surfaces that require sterilizing. The device allows air to be drawn across the lamps and sterilized. The device however, does not provide cleaning of odor causing bacteria in shoes. Further, none of the devices

Accordingly, difficulties in the field of eliminating odor-causing bacteria in the interior of shoes remain. Further, existing solutions fail to address particular deficiencies that confront consumers seeking alternatives to the existing art and a solution to advancing the life of shoes and in eliminating embarrassing foot odor when shoes are removed in public spaces. The present invention addresses these shortcomings.

### SUMMARY OF THE INVENTION

The present invention is directed to methods and an improved device for eliminating odor-causing bacteria, fungus and other microbes that cause odor to proliferate in the interior of shoes. The device provides an ultra-violet (UV) light source that is fitted within a framework designed to fit perfectly inside any shoe, with different size frames designed to fit differently sized shoes. The frame of the device is open thus allowing a maximum amount of UV light to permeate throughout the interior of the shoe, killing all odor-causing microbes therein. The framework of the device contours the interior of the shoe and is prepared from a durable metallic allow. Alternatively, the device framework can be prepared from plastic or other synthetic polymers. The device also provides a UV light source that is fitted in the central section of the metallic framework and can be battery powered or powered by an electrical source.

The present invention is also directed to a mobile sanitizing and deodorizing device, that includes a housing shaped substantially to fit within the interior of a shoe, the housing prepared with cross members and a framework. The housing also employs an ultra-violet (UV) light source configured within the housing in which the cross members and framework are designed to allow the UV light source to illuminate the interior of the shoe with UV light. The light source also employs a power supply that is enabled to allow a user to adjust the intensity of the UV light source manually, the light source, when powered on, acts to substantially deodorize the interior of the shoe by killing the odor-causing microbes therein.

### BRIEF DESCRIPTION OF THE DRAWINGS

FIG. 1 depicts the basic configuration of invention. As illustrated, the invention is shown from a side angle view wherein the device includes a UV light source housed within a rigid outer shell which allows the light to escape and radiate to the interior of a shoe.

FIG. 2 depicts the underside of the invention. As shown, the device is oval shaped on the front end and flat on the back end in order to fit snugly inside the interior of a typical shoe or sneaker.

FIG. 3 depicts an exploded view of the device whereupon assembly of the device is illustrated. Specifically, the UV light source is fitted into the outer rigid shell by means of, for example, a screw that secures the light source to the shell.

FIG. 4 illustrates a side profile view of the device that has been placed within the interior of a sneaker. The illustration demonstrates the snugness by which the invention fits within the interior of a typical shoe in order to illuminate the shoe interior with UV light.

FIG. 5 illustrates the device in a preferred embodiment in which two devices are attached to an electrical cord and electrical source to provide power to the light source of the device.

### DETAILED DESCRIPTION OF THE INVENTION

The present invention provides an apparatus and methods that allow a user to effectively kill odor-causing microbes that populate the interior of ordinary shoes thus, eliminating the embarrassing foot odor that accompanies routine use of shoes by the user. The portability of the device also allows the device to be used on a regular basis whether the user is a frequent travelor or is situated primarily in a home setting. To kill odor-causing bacteria the device provides a metallic, metallic alloy, plastic or other appropriate material to form a framework designed and shaped to fit easily within the interior of a shoe. The device is also fitted with a source of ultra-violet (UV) light in the central portion of the framework so that when the lighting element is turned on, the interior of the shoe is illuminated with UV radiation to a sufficient degree to kill the bacteria contained therein. As the interior of the shoe is illuminated with UV light, over the course of a few hours, the odor-causing microbes present within the shoe, are killed.

The UV source is either battery powered or powered with electricity depending on the needs of the user. The device provides narrow framework trusses so to allow the maximum amount of UV illumination to permeate throughout the interior of the shoe as possible. The light source can be any bulb type that provides a source of UV radiation.

It is therefore, a primary object of the present invention to provide an improved method and device for killing odor-causing microbes including bacteria, fungus and others in the interior of a shoe or pair of shoes, so to eliminate the embarrassing foot odor associated with prolonged and continued use of shoes in the course of a wearer's day and evening hours.

In a preferred embodiment of the invention, a semi-flexible, break-resistant material is employed as forming the framework of the device. The flexibility of the framework allows a user to employ the device in different sized shoes and to conform to different types of shoes. In this embodiment, the framework is composed of a flexible plastic, semi-rigid latex, or other appropriate synthetic material that allows for flexibility in the framework.

In yet another preferred embodiment of the invention, a rigid, break-resistant material is employed as the framework for the device. The rigidity of the embodiment acts to maintain the shape of the shoe as the device kills bacteria and other microbes and deodorizes the interior of shoe. An added benefit to this embodiment allows the user to employ the use of the device during periods of time even with the UV light source is not activated. In so using the device, the user is able to keep shoes in good shape for continued, longer-term use, thus increasing the life of a typical pair of shoes. A shoe shaping device such as embodied by the present invention provides yet another benefit of the claimed device that isn't addressed by the prior art.

As envisioned herein, the invention employs a UV light source situated within a rigid or semi-flexible framework that fits snugly within the interior of a shoe in order to kill microbes contained therein, in order to deodorize the shoe. The device is manufactured in different sizes in order to fit naturally within different sized shoes. Likewise, the device is manufactured in order to fit different style shoes. For example, a device of the invention is manufactured to fit within the interior of a woman's high heel shoe. Accordingly, the device in such a case is manufactured with an angled framework so that the device can be situated into a typical high heel shoe.

In yet another embodiment, the device is manufactured to fit into the interior of a shoe with a flat heel. For example, the device is manufactured to fit within the interior of a closed-toed sandal or slipper. In such a case, the device is manufactured to have a flat framework work so that it fits easily within the interior of the sandal or slipper. In yet another embodiment, the device is manufactured to fit within a shoe with a medium heel and narrow interior. For example, the device is manufactured to fit within the interior of a woman or man's long boot. In such a case, the device is manufactured with a moderately angled framework, with the framework being narrower than a typical width, in cases in which the boot is designed to fit a narrow foot.

The outer shell will also be adjustable in order to allow easy use in a variety of different shoes. For example, the adjustable outer shell can be completely compacted so that it occupies very little space.

The outer shell can be assembled in a variety of configurations as illustrated herein. The outer shell can be assembled and prepared from any materials as known in the art including but not limited to aluminum, thermoplastics, steel or other rigid metals that can be machined into the shell described.

The present invention provides technology to neutralize and eliminate harmful bacteria and germs that lead to disease and sickness. It provides pathogen killing in order to remove odors, kill fungus and optimally sterilize away germs/bacteria with UV-C germicidal radiation.

Ultraviolet light (UV) with wavelength shorter than 300 nanometers is known to be significantly effective in killing microorganisms. An effective sterilizing range of UV is within the C bandwidth (UVC—253.7 nm). This range—between 100 nm and 280 nm (4.43-12.4 eV)—is called germicidal UV bandwidth or UVC. Germicidal ultraviolet (UVC) is used for UV disinfection and inactivates microorganisms by disrupting their cellular membranes and by damaging their DNA or RNA. If cellular processes are disrupted because of DNA damage, the cell cannot carry out its normal functions and cannot reproduce.

As envisioned herein, the device of the invention is shaped to fit within the interior of various shoe styles including for example, high heels, sneakers, dress or sandal type shoes.

The invention is powered either by battery power, which can be rechargeable or alternatively, from an electrical power source via power cord. The framework and body of the device is manufactured from any appropriate materials as known in the art including for example, metallic alloys, semi-rigid plastics or other materials that can be foreseen by those skilled in the art of manufacturing.

The device of the invention emits light in an effective sterilizing wavelength range within the C bandwidth of between 100 and 280 nm but can alternatively span a range of between, 200 and 280 nm, 240-270 nm, 250-260 nm, 252-254 nm and other wavelength values within these ranges, including most optimally at 253.7 nm. The sanitizing and deodorizing device of the invention also employs a UV dose in the range of between 15,000 to 55,000 μWs/cm². The device also employs a UV light source that allow a user to manually adjust the amount of time the UV source irradiates the interior of a shoe or other article of clothing.

As envisioned herein, the UV light source is powered via a timer that allows a user to manually adjust the amount of time the UV source irradiates the interior of a shoe or other article of clothing.

Ozone is the second most powerful sterilant in the world. One function of ozone is to destroy bacteria, viruses and odors. Ozone also occurs commonly in nature as a result of lightning strikes during thunderstorms. The “fresh, clean, spring rain ” smell that is apparent after a storm results from nature's creation of ozone. Ozone acts 3000 times faster than chlorine as a bactericide, and it is the strongest oxidant commercially available for air & water treatment.

One of the best features of ozone is its ability to control odors by odor producing pathogens such as bacteria, fungi and viruses. Ozone is also known to be one of the most powerful and effective eliminators of smoke and a myriad of other odors. Ozone generating machines utilize ozone to destroy tobacco smoke and its inherent impurities plus odors from pets, cooking, mold, mildew and other household sources. As envisioned herein, the invention employs natural ultraviolet light with ozone function to kill harmful germs and bacteria that can lead to foot fungus, disease, infection and unpleasant odors.

The present invention also provides a method of manufacturing the sanitizing and deodorizing device described herein. Specifically, the device is manufactured to include a housing shaped substantially to fit within the interior of a shoe. The housing includes cross members and a framework, the housing containing within it, an ultra-violet (UV) light source. As contemplated herein, the cross members and framework are designed to allow the UV light source to illuminate the interior of the shoe with UV light in order to kill pathogenic microbes therein. The light source includes a power supply that is enabled to allow a user to adjust the intensity of the UV light source manually in order to substantially deodorize the interior of the shoe by killing the odor-causing microbes therein.

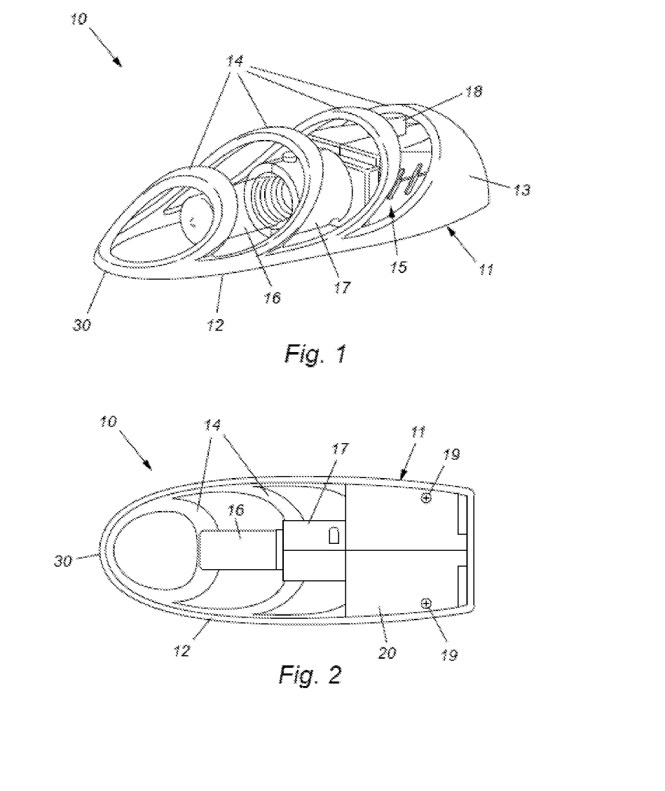

Turning now to the substance of FIGS. 1 to 5 and the preferred embodiments of the invention. FIG. 1 provides a side angle view perspective of a preferred embodiment of the present invention. Specifically, the device 10 is illustrated with several elements including a shoe sanitizing cover 11, a lower rail 12, a side rail cover and several cross members 14 that encase the UV light bulb 16 and bulb housing 17 and electrical unit which provides power to the device 15.

The Figure also illustrates an adjustment dial 18 that allows a user to adjust the degree of luminosity radiated into the interior of the shoe. Also illustrated is the front tip of the shoe sanitizer 30 that allows the device to fit snugly within the interior of the shoe.

FIG. 2 is a view of the sanitizing device 10 from underneath. As shown, the sanitizer includes an oval shaped tip 30 for snug fit into the interior of a shoe with several cross members 14 that form a rigid framework for the device but which also allow UV light to pass through to the shoe. The UV light as envisioned herein emits light radiation in the wavelength range and effective sterilizing range of UV is within the C bandwidth (UVC—253.7 nm). More broadly, the light source provides a sterilizing wavelength range within the C bandwidth of between 100 and 280 nm but can alternatively span a range of between, 200 and 280 nm, 240-270 nm, 250-260 nm, 252-254 nm and other wavelength values within these ranges, including most optimally at 253.7 nm. The sanitizing and deodorizing device of the invention also employs a UV dose in the range of between 15,000 to 55,000 μWs/cm². This range—between 100 nm and 280 nm (4.43-12.4 eV)—is typically known as the germicidal UV bandwidth or UVC. The illustration also demonstrates the open framework of the bottom of the shoe sanitizer 20 that allows UV light to pass onto the bottom of a shoe uninterrupted by the framework. Assembly of the device includes a lower, bottom rail 12, the shoe sanitizing cover 11 and the UV bulb 16 and electrical source unit 17. Assembly also includes the use of securing means such as for example, screws 19 that attach the UV light electrical unit to the shoe cover 11.

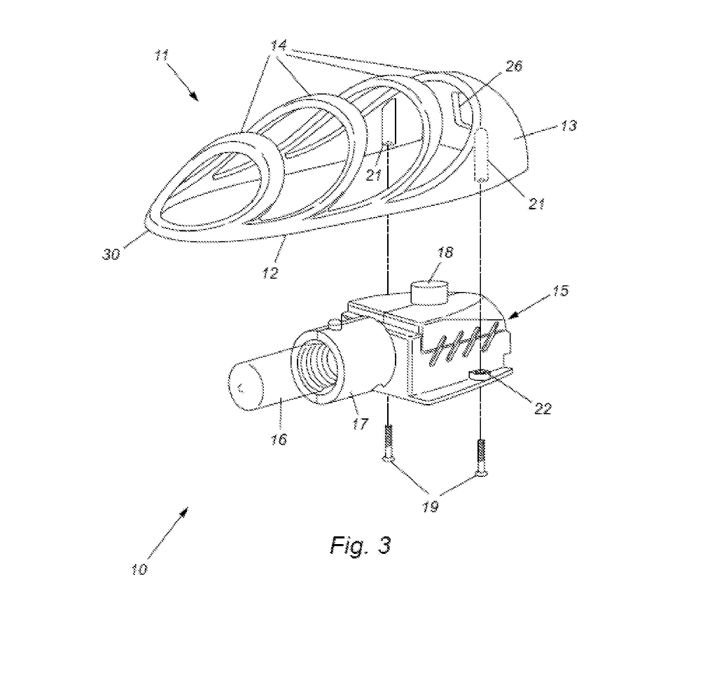

FIG. 3 illustrates the assembly of the device 10. In a preferred embodiment the device is secured in primarily two sections, a shoe sanitizer cover 11 and an electrical unit and power supply 15. Encompassed within this embodiment are elements associated with each. For example, the shoe sanitizing cover 11 includes several cross members 14 that allow light to pass through to the interior of the shoe. The shoe sanitizing cover also includes rigid framework to form the cover including a lower rail 12, a side rail cover 13 and a tip 30 to allow a snug fit. As shown, the cover also provides an opening 26 so that UV light can pass through to the rear of the interior of the shoe. The shoe cover also includes attachment points 21 so to secure screws 19 to the electrical unit thereto.

The electrical unit 15 includes several elements including a UV bulb 16, a bulb housing 17 and an adjustment dial 18 that allows the user to adjust the intensity by which the interior is irradiated with UV light. Screws 19 secure the electrical unit at attachment points 22 to the shoe cover 11.

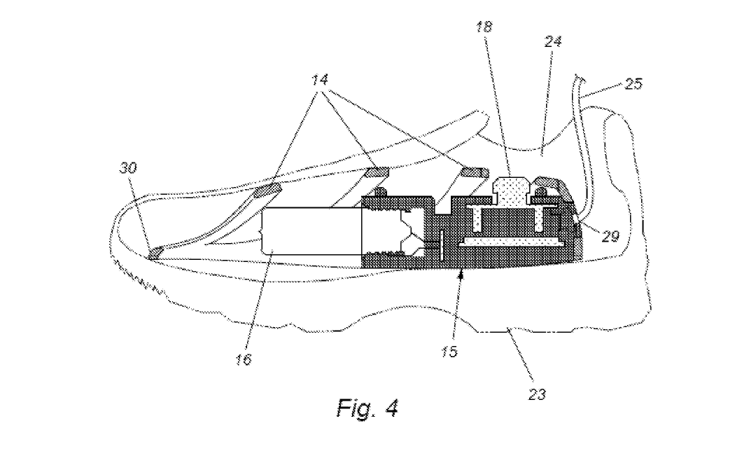

FIG. 4 illustrates the device as it is placed within the interior of a shoe 23 through the shoe opening 24. In a preferred embodiment the cross members 14 form a rigid framework at the upper section of the shoe interior with the tip of the device 30 allowing for a full extension of the device within the interior of the shoe. In this preferred embodiment, the electrical unit 15 and UV bulb 16 are powered by an electrical cord adaptor 25 with the electrical cord attached to the electrical unit to supply power thereto 29. In other preferred embodiments, the electrical cord adaptor is not required and the UV light source is powered by rechargeable means or by battery power.

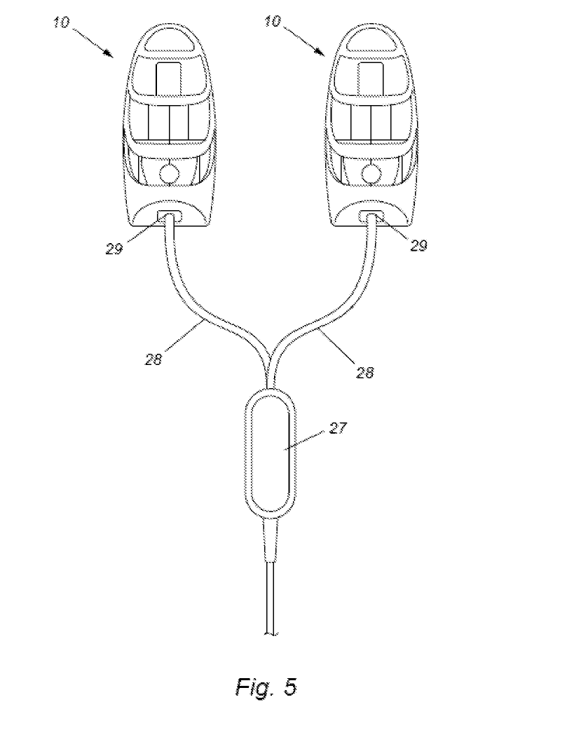

FIG. 5 illustrates an overhead view of a preferred embodiment of the device. As shown, two shoe sanitizer assemblies 10 are provided. Electrical power is supplied by means of an electrical adaptor 27 and electrical adaptor cords 28 that supply power to the sanitizer assemblies via an adaptor attachment point 29 on each of the devices. As described, optionally, the devices can be powered by means of a rechargeable battery or by a typical battery source.

In a preferred embodiment of the invention the shoe sanitizer can be configured in either a rigid outer shell or a semi-rigid framework in order to accommodate different style shoes. Further, the outer shell, can be manufactured in different sizes to accommodate different sized shoes.

To prepare the rigid or semi-rigid framework, several manufacturing methods commonly known in the art are employed. For example, cast injection molding can be employed wherein an aluminum alloy is used to form the sanitizer framework. Such methods of manufacture allow for mass production as well.

For manufacturing the rigid outer shell, materials such as those known in the art may be utilized. For example, aluminum or aluminum alloys are used however, other appropriate materials including for example rigid plastics and polymers may be used likewise. The framework can also be prepared using a rigid material such as silicone based polymers or Teflon in which the material is molded at melting point temperatures and cooled to produce the necessary size.

Injection molding is accomplished by large machines called injection molding machines. Resin is fed to a machine through what is called a hopper. Colorants are fed to the machine directly after the hopper. The resins enter the injection barrel by gravity though the feed throat. Upon entrance into the barrel, the resin is heated to the appropriate melting temperature.

The resin is injected into the mold by a reciprocating screw or a ram injector. The reciprocating screw offers the advantage of being able to inject a smaller percentage of the total shot (amount of melted resin in the barrel). The ram injector must typically inject at least 20% of the total shot while a screw injector can inject as little as 5% of the total shot.

Certain advantages can be obtained in utilizing the present invention. Foremost, the invention allows a user to effectively increase the life of the shoes significantly over shoes that are not sanitized in such a manner. The cost savings of shoes over the life of a user can be significant and the device of the invention allows significant savings in the purchase of new shoes.

Benefits of the present invention over the prior art also include elimination of bad, foul odors that can cause embarrassment in public settings. Regular use of the device will kill the odor causing bacteria that proliferate within the interior of shoes thus, eliminating the odor these bacteria produce.

Although the invention has been described with reference to the above examples, it will be understood that modifications and variations are encompassed within the spirit and scope of the invention. Accordingly, the invention is limited only by the following claims.

What is claimed is:

1. A sanitizing and deodorizing device, comprising a housing shaped substantially to fit within the interior of a shoe, the housing comprised of cross members and a framework, the housing further comprising an ultra-violet (UV) light source configured within the housing wherein, said cross members and framework are designed to allow the UV light source to substantially illuminate the interior of the shoe with UV light, the light source further comprising an electrical unit and power supply that is enabled to allow a user to adjust the intensity of the UV light source manually, the light source, when powered on, acting to substantially deodorize the interior of the shoe by killing the odor-causing microbes therein.

2. The sanitizing and deodorizing device of claim 1 wherein, the device is shaped to fit within the interior of various shoe styles.

3. The sanitizing and deodorizing device of claim 2 wherein, said various shoe styles include high heels, sneakers, dress or sandal type shoes.

4. The sanitizing and deodorizing device of claim 1 wherein the device is powered by a battery.

5. The sanitizing and deodorizing device of claim 4 wherein the battery is rechargeable.

6. The sanitizing and deodorizing device of claim 1 wherein the housing is comprised of a rigid metallic alloy.

7. The sanitizing and deodorizing device of claim 1 wherein the housing is comprised of a semi-rigid plastic.

8. The sanitizing and deodorizing device of claim 1 wherein the UV light source emits light in an effective sterilizing wavelength range within the C bandwidth of between 100 and 280 nm.

9. The sanitizing and deodorizing device of claim 8 wherein the effective sterilizing wavelength is in a range of between 200 and 280 nm.

10. The sanitizing and deodorizing device of claim 9 wherein the effective sterilizing wavelength is in a range of between 240 and 270 nm.

11. The sanitizing and deodorizing device of claim 10 wherein the effective sterilizing wavelength is in a range of between 250 and 260 nm.

12. The sanitizing and deodorizing device of claim 11 wherein the effective sterilizing wavelength is in a range of between 252 and 254 nm.

13. The sanitizing and deodorizing device of claim 12 wherein the effective sterilizing wavelength is 253.7 nm.

14. The sanitizing and deodorizing device of claim 1 wherein the UV light source is powered via a timer allowing a user to manually adjust the amount of time the UV source irradiates the interior of said shoe.

15. The sanitizing and deodorizing device of claim 1 wherein the UV light source delivers a UV dose in the range of between 15,000 to 55,000 μWs/cm².

16. The sanitizing and deodorizing device of claim 1 as shown in FIGS. 1-5.

17. A method of manufacturing a sanitizing and deodorizing device comprising a housing shaped substantially to fit within the interior of a shoe, the housing comprised of cross members and a framework, the housing further comprising an ultra-violet (UV) light source configured within the housing wherein, said cross members and framework being designed to allow the UV light source to illuminate the interior of the shoe with UV light, the light source further comprising an electrical unit and power supply that allows a user to adjust the intensity of the UV light source manually, the light source, when powered on, acts to substantially deodorize the interior of the shoe by killing the odor-causing microbes therein.

18. The method of claim 17 wherein the ultra-violet (UV) light source emits light in an effective sterilizing wavelength of between 250 and 260 nm.

19. The method of claim 18 wherein the ultra-violet (UV) light source emits light in an effective sterilizing wavelength 253.7.

20. The method of claim 17 wherein the sanitizing and deodorizing is manufactured to conform with illustrations as shown in FIGS. 1-5.
